(19) United States
(12) Patent Application Publication
(10) Pub. No.: US 2017/0036204 A1
(43) Pub. Date: Feb. 9, 2017

(54) SAMPLE COLLECTION UNIT

(71) Applicant: Siemens Healthcare Diagnostics Inc., Tarrytown, NY (US)

(72) Inventors: Amy Zercher, Nappanee, IN (US); Jennifer Goldsburg, Vandalia, MI (US)

(73) Assignee: Siemens Healthcare Diagnostics Inc., Tarrytown, NY (US)

(21) Appl. No.: 15/303,903

(22) PCT Filed: Apr. 13, 2015
(86) PCT No.: PCT/US15/25558

§ 371 Date: Oct. 13, 2016

Related U.S. Application Data
(60) Provisional application No. 61/984,352, filed on Apr. 25, 2014.

(51) Int. Cl. B01L 3/00; B01L 9/00; G01N 21/78; A61B 10/00

(52) U.S. Cl. CPC B01L 3/5023; A61B 10/007; B01L 9/52; B01L 3/5082; G01N 21/78; A61B 2010/0006; B01L 2300/0825; B01L 2300/0832

(57) ABSTRACT

An insert for a sample collection tube includes a generally hollow tubular body insertable into the sample collection tube. The tubular body is open at least at one end. The tubular body of the insert has an internal cross-sectional area dimensioned to accommodate a width of test strip which comprises a plurality of test areas arranged along its length. The internal cross-sectional area of the tubular body is further dimensioned such that volume of 2.5 mL of a liquid sample inside the insert occupies a height which is at least sufficient to wet all of the test areas of the test strip when the strip is inserted lengthwise into insert containing the liquid sample.

The subject application claims benefit under 35 USC §119(e) of U.S. provisional Application No. 61/984,352, filed Apr. 25, 2014. The entire contents of the above-referenced patent application are hereby expressly incorporated herein by reference.

### BACKGROUND

Field

Embodiments illustrated herein relate to collection of a biological sample, such as urine.

Description of the Related Art

Biological samples are generally collected in standardized sample collection tubes. In particular, the collection tubes may have standardized dimensions adapted to the size of standardized racks, for example as provided in storage units or in test equipment.

In the field of urinalysis, for testing of a urine sample, a so-called dip-and-read test strip may be used. Such a test strip usually has one or more test areas, also called reagent pads, and each test area is capable of undergoing a color change in response to contact with a liquid specimen, which in this case is a urine sample. The liquid sample usually contains one or more analytes of interest. The presence and concentrations of these analytes of interest in the sample are determinable by an analysis of the color changes undergone by the reagent pads. Usually, this analysis involves a color comparison between the reagent pad and a color standard or scale. The comparison may be done manually or using test equipment, including, for example, a spectrophotometer.

Testing of a urine sample requires wetting the reagent pads of the dip-and-read test strip. This is typically carried out by collecting the urine sample in a standardized sample collection tube, also known as a urinalysis tube, and then dipping the test strip into the collection tube to wet the reagent pads.

A standard sample collection tube for urine dip test strips requires 10-12 mL of the sample fluid (i.e, urine) for manual or machine dip to be efficient, that is, to wet all of the reagent pads of the test strip. However, many patients, for example, neonates, cannot provide 10 mL of urine consistently.

Current practice involves repeated collection of urine samples from the patient. However, a portion of the sample will be aged in this case. Samples from low volume patients can be obtained at a later time in the same day, but this uses hospital resources or can delay or postpone or require repeated office visits. Alternately, smaller tubes can be obtained, but they do not typically fit well in the standardized racks of storage units or automated test equipment.

### SUMMARY

The summary is provided to introduce a selection of concepts that are further described in the detailed description. This summary is not intended to identify key or essential features of the claimed subject matter, nor is it intended to be used as an aid in limiting the scope of the claimed subject matter.

Briefly, embodiments described in the present disclosure relate to collection of a biological sample, such as urine, and in particular, to an insert for a sample collection tube.

In a first aspect, an insert for a sample collection tube is provided. The insert includes a generally hollow tubular body insertable into the sample collection tube. The tubular body is open at least at one end. The tubular body of the insert has an internal cross-sectional area dimensioned to accommodate a width of test strip which comprises a plurality of test areas arranged along its length. The internal cross-sectional area of the tubular body is further dimensioned such that volume of 2.5 mL of a liquid sample inside the insert occupies a height which is at least sufficient to wet all of the test areas of the test strip when the strip is inserted lengthwise into insert containing the liquid sample.

In a second aspect, a urine sample collection kit is provided. The urine sample collection kit includes a collection tube and an insert. The insert comprises a generally hollow tubular body with an open end. The tubular body is narrower than the collection tube and insertable into the collection tube. The tubular body of the insert has an internal cross-sectional area which is dimensioned with respect to the collection tube such that a volume of 2.5 mL of a urine sample inside the insert occupies a height which is at least equal to a height that would occupied by a 10 mL volume of the urine sample within the collection tube.

In a third aspect, a urinalysis kit is provided. The urinalysis kit comprises a collection tube and an insert comprising a generally hollow tubular body narrower than the collection tube and insertable into the collection tube. The kit also includes a test strip comprising a plurality of analyte sensitive test areas, the test areas being arranged along a length of the test strip. The tubular body of the insert has an internal cross-sectional area dimensioned to accommodate the width of the test strip, such that the test strip is insertable lengthwise into the tubular body. The internal cross-sectional area of the tubular body is further dimensioned such that volume of 2.5 mL of a urine sample inside the insert occupies at least a height which is sufficient to wet all of the test areas of the test strip, when the strip is inserted lengthwise into the insert containing the urine sample.

In a fourth aspect, a urine sample container is provided. The container includes a unitary elongated body comprising an open first end configured to receive a urine sample and a closed second end. The unitary elongated body has an outer surface defining an outer cross-sectional area and an internal cavity defining an internal cross-sectional area narrower than the outer cross-sectional area. The internal cross-sectional area of the cavity is dimensioned to accommodate a width of test strip which comprises a plurality of test areas arranged along its length. The internal cross-sectional area of the cavity is further dimensioned such that volume of 2.5 mL of the urine sample in the cavity of the container occupies a height which is at least sufficient to wet all of the test areas of the test strip when the strip is inserted lengthwise into the cavity of the container containing the urine sample.

### BRIEF DESCRIPTION OF THE DRAWINGS

A more complete appreciation of the present disclosure and many of the attendant advantages thereof will be readily understood by reference to the following detailed description when taken in conjunction with the accompanying drawings, in which:

FIG. 1 illustrates a perspective view of a sample collection tube in accordance with one embodiment;

FIG. 2 illustrates a test strip in accordance with one embodiment;

FIG. 3 illustrates a perspective view of an insert in accordance with one embodiment;

FIG. 4 illustrates a perspective view of a sample collection sample kit in accordance with one embodiment, showing an insert positioned within a collection tube for receiving a sample;

FIG. 5 illustrates a perspective view of a sample kit in accordance with one embodiment, showing a test strip inserted into an insert positioned within a collection tube; and

FIGS. 6A-6D illustrate steps of manufacturing an insert from a pipette in accordance with an exemplary embodiment.

### DETAILED DESCRIPTION

The following detailed description refers to the accompanying drawings. The same reference numbers in different drawings may identify the same or similar elements.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements, but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the inventive concept. This description should be read to include one or more and the singular also includes the plural unless it is obvious that it is meant otherwise.

Further, use of the term “plurality” is meant to convey “more than one” unless expressly stated to the contrary.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

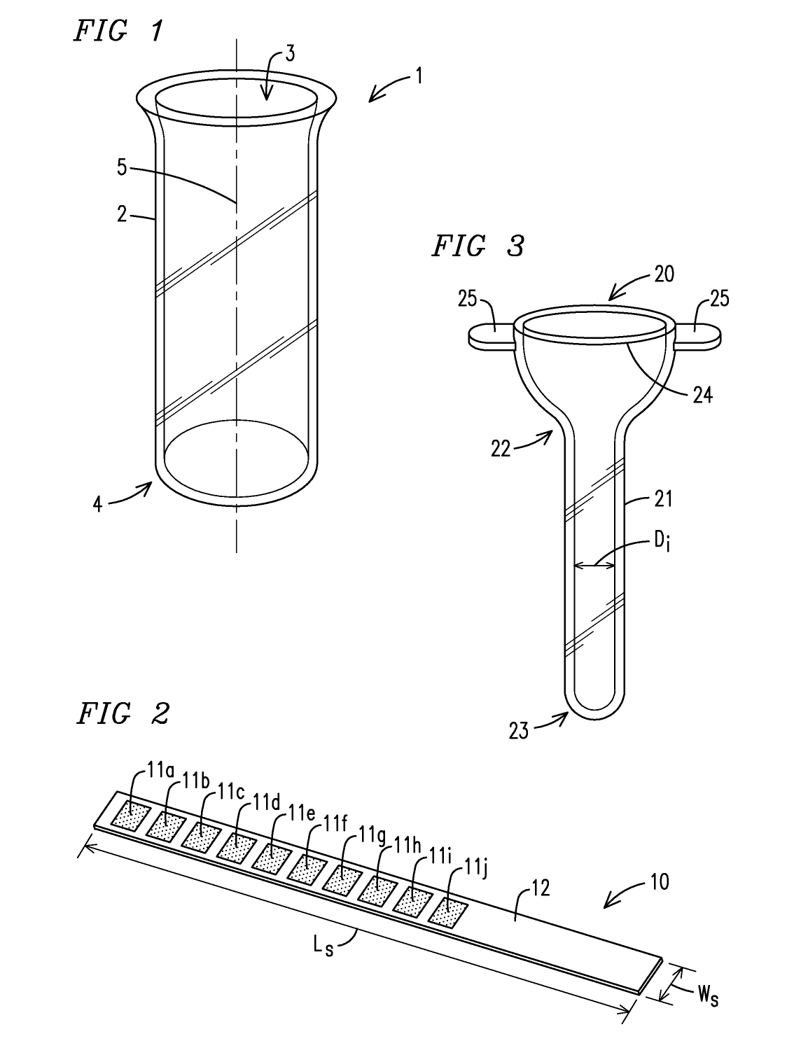

Turning now to FIG. 1, a sample collection tube 1 is illustrated. In the illustrated embodiment, the tube 1 is a standard urinalysis tube that may be used for collecting a urine sample therein. An exemplary embodiment of such a urinalysis tube is available under the trademark ‘Novus’. The sample collection tube 1 has a hollow elongated body 2, which is open at a first end 3 and closed at a second end 4. The elongated body 2 may have a circular cross-section about a central axis 5. As per the currently used urinalysis procedure, the urine sample from a patient is collected inside the standard sample collection tube 1, and stored in standardized racks specially designed to receive such tubes.

The subsequent testing of the urine sample may involve a test device, for example a dip-and-read test strip, also referred to as urine dip strip. Illustrative of dip-and-read test devices currently in use are products available from Siemens Healthcare Diagnostics Inc., under the trademark MULTISTIX, and others.

An exemplary test strip 10 is shown in FIG. 2. The illustrated strip 10 is generally planar, having a rectangular shape with a length Ls and a width Ws. For example, for a MULTISTIX® 10SG strip, the length Ls is approximately 4.25 inches, and the width Ws is approximately 0.2 inches. The test strip 10 comprises a non-reactive surface 12, which is typically white in color. Arranged along the non-reactive surface 12, along the length direction, is an array of test areas 11-a-j.

In the illustrated embodiment, the test areas 11a-j are embodied as reagent pads, and each test area is capable of undergoing a color change in response to contact with a liquid specimen, such as a biological sample. The liquid sample, in this case, the urine sample, usually contains one or more analytes of interest. The presence and concentrations of these analytes of interest in the sample are determinable by an analysis of the color changes undergone by the reagent pads. Usually, this analysis involves a color comparison between the reagent pad and a color standard or scale. The comparison may be done manually by a skilled technician or by automated test equipment.

In one example, a reflectance spectroscope may be used to analyze analytes of interest applied to the reagent pads. A spectrophotometer determines the color of a sample applied to one or more of the reagent pads 11a-j disposed on the white non-reactive surface 12 by illuminating the pad and taking a number of reflectance readings from the respective pad 11a-j, each having a magnitude relating to a different wavelength of visible light. Strip reading instruments may employ a variety of area array detection read-heads utilizing CCD (charge-coupled device), CID (charge-injection device) or PMOS detection structures for detecting color changes to the reagent pads. The color of the sample on the reagent pad may then be determined based upon the relative magnitudes of red, green and blue reflectance signals.

A spectrophotometer may be used, for example, to perform a number of different urinalysis tests utilizing the test strip 10 on which a number of different reagent pads 11a-j are disposed. Each reagent pad 11a-j is provided with a different reagent which causes a color change in response to the presence of a certain type of constituent in a sample such as leukocytes (white blood cells), red blood cells, glucose, bilirubin, urobilinogen, nitrite, protein, ketone bodies, or other analytes of interest. The color developed in a particular analyte defines the characteristic discrete spectrum for absorption of light for that particular analyte. For example, the characteristic absorption spectrum for color-developed glucose falls within the upper end of the blue spectrum and the lower end of the green spectrum. In the illustrated embodiment, ten distinct reagent pads 11a-j are provided on the test strip 10.

A standardized sample collection tube would require a high volume of the sample in order to wet a required number of test areas of the test strip. For example, in case of the exemplary urine sample collection tube illustrated above, a volume of about 10-12 mL of urine sample is necessary to occupy the required height in the collection tube so as to wet all of the test areas of the exemplary test strip when the test trip is dipped into the urine sample contained in the sample collection tube. If a patient is unable to provide such a high volume of urine sample at a time, multiple samples may need to be collected at different points in time.

Embodiments of the inventive concept illustrated below provide an inexpensive and disposable insert for a sample collection tube, which will contain a small of the sample enough to occupy the height necessary to wet all of the test areas of the test strip.

An exemplary insert 20 is illustrated in FIG. 3. The insert 20 may be configured as an insert for the exemplary urine sample collection tube 1 described above. However, the inventive concepts underlying the exemplary embodiments may be utilized in a variety of different applications involving collection of various biological or chemical samples. The insert 20 has a hollow tubular body 21 having an open end 22. The tubular body 21 is narrower than the sample collection tube 1 illustrated in FIG. 1. The tubular body 21 is insertable into the collection tube for sample collection, whereby tubular body 21 can be positioned within the sample collection tube to receive the liquid sample (in this example, urine sample) via its open end 22. In the illustrated embodiment, the open end 22 is integrally connected to a funnel-shaped portion 24 via which the urine sample may be poured into the insert 20. In alternate embodiments, the funnel shaped portion 24 may be omitted from the structure of the insert 20, and the liquid ample may be poured into the insert by way of a dropper or an external funnel. The other end 23 of the insert 20 opposite to the open end 22 may be sealed off, to prevent the liquid sample from leaking out of the insert 20 when the insert 20 is positioned within the sample collection tube 1 for sample collection. In other embodiments, the end 23 may be left open. Additional sealing mechanism may be provided in this case to the sample collection tube and/or the insert, to ensure that the liquid sample does not leak from the bottom of the insert when positioned in the sample collection tube.

In the illustrated embodiments, the tubular body 21 is shown to have a circular cross-section. However, alternate cross-sectional shapes, such as rectangular, square, trapezoidal, triangular or polygonal shapes may be used. An underlying feature in the above embodiments is that the internal cross-sectional area of the tubular body 21 is dimensioned to accommodate the width of the test strip, such that the test strip is insertable lengthwise into the tubular body 21. Furthermore, internal cross-sectional area of the tubular body is dimensioned such that volume of 2.5 mL of a urine sample inside the insert occupies at least a height which is sufficient to wet all of the test areas of the test strip, when the strip is inserted lengthwise into the insert containing the urine sample.

In other words, the illustrated insert 20 is structurally configured so as to be, on one hand, wide enough to accommodate the width of the test trip, and on the other hand, narrow enough to cause the liquid inside to occupy a desired height for an efficient dipping of the test strip on the other hand. A volume of 2.5 mL may be consistently obtained from most patients, including neonates, whereby repeated sample collection may be avoided.

As an example, the insert 20 may be dimensioned such that the internal diameter Di of the tubular body 21 is 0.2-0.3 inches. In particular, in the illustrated example, an internal diameter of 0.25 inch of the tubular body 21 would accommodate the width of the test strip 10 exemplified in FIG. 2 and would ensure that a 2.5 mL volume of urine sample rises up to a height of 4.5 inches, which would be enough to wet all of the test areas of the exemplary test strip described above. In other examples, the height occupied by the volume of 2.5 mL of the liquid sample in the insert may vary from 4.25 to 4.75 inches However, the inventive concepts are not meant to be limited by the specific dimensions exemplified.

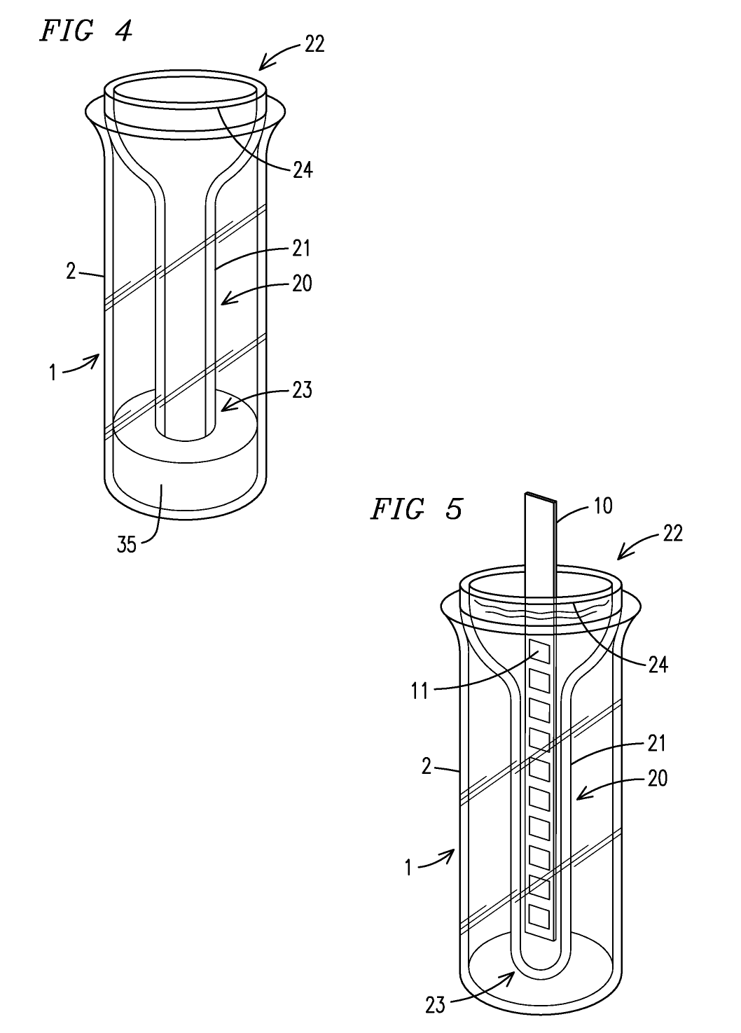

FIG. 4 illustrates a sample collection kit 30 according to one aspect, including a sample collection tube 1 with an insert 20 positioned within the sample collection tube 1 with its open end 22 facing upwards for receiving the liquid sample. As per embodiments of the inventive concept, instead of filling up the entire sample collection tube 1, the liquid sample is only contained within the insert 20. Herein, a volume of 2.5 mL would be enough to occupy the necessary height to wet all the reagent pads of a test strip.

In an exemplary embodiment, an inventive insert may have a structure wherein the tubular body of the insert has an internal cross-sectional area which is dimensioned with respect to the exemplary standardized collection tube of FIG. 1, such that a volume of 2.5 mL of a urine sample inside the insert occupies a height which is at least equal to a height that would occupied by a 10 mL volume of the urine sample within the sample collection tube. This would ensure that when a test strip, such as that exemplified in FIG. 2 is dipped into the insert containing the urine sample, all of the reagent pads would be wetted. Furthermore, such an insert would allow suction probes of automated test equipment to collect sufficient sample without air intrusion.

In another aspect, the above illustrated concepts may be integrated to provide a stand alone urine sample collection container. In this case the container may have a unitary elongated body with an outer surface corresponding with an outer cross-sectional area dimensioned to correspond to that of a standardized urine sample collection tube, such as the tube 1 in the illustrated embodiments. This will ensure that the container is usable in standardized racks in storage units and test equipment. The container may have an internal cavity with an internal cross-sectional area narrower than the outer cross-sectional area and specifically configured for urine dip-and-read strips, for example, of the type illustrated above. For this purpose, the internal cross-sectional area of the cavity may be dimensioned to accommodate a width of test strip which comprises a plurality of test areas arranged along its length. The internal cross-sectional area of the cavity may be further dimensioned such that volume of 2.5 mL of the urine sample in the hollow inside of the container occupies a height which is at least sufficient to wet all of the test areas of the test strip when the strip is inserted lengthwise into the hollow inside of the container containing the urine sample.

In one embodiment, the above described unitary urine sample collection container may be realized by forming the parts 20 and 1 described in FIG. 4 integral to each other, as opposed to using a separate insert 20 for a standardized urine sample collection tube 1. In another embodiment, the unitary urine sample collection container may be realized by forming a urine collection tube with a thick wall, whereby the outer dimensions of a standardized urine sample collection tube are retained, while the thickness of the wall is configured to provide the internal cross-sectional dimensions in line with the inventive concepts illustrated herein.

FIG. 5 illustrates an exemplary urinalysis kit including a sample collection tube 1, an insert 20 positioned within the sample collection tube 1 and a test strip 10 inserted lengthwise into the insert 20. In this embodiment, a volume of 2.5 mL of a urine sample is shown to wet all of the reagent pads 11 of the test strip 10.

As shown in the embodiment shown in FIG. 3, as an optional feature, the insert 20 may provided with a support structure 25 for positioning the insert 20 centrally within the sample collection tube. In this embodiment, the support structure is embodied as one or more radial ribs attached to an outer surface of the funnel-shaped portion 24. However, alternate designs may be conceived. For example, the funnel portion 24 of the insert 20 may be designed to be wider than the sample collection tube 1, whereby the inert 20 may be supported in position centrally within the sample collection tube 1 by way of the funnel-shaped portion 24. Furthermore, support structures 25 may be alternately or additionally provided on the outer surface of the tubular body 21. In a still further embodiment as illustrated in FIG. 4, alternate to or in addition to the support structures in the insert 20, the sample collection tube 1 may be optionally provided with positioning structures 35 for centrally receiving the insert 20 within the sample collection tube 1. An underlying feature the illustrated embodiments is that the sample is confined to the central axis of the sample collection tube 1 so that probes from automated test equipment encounter no interference from the walls of the insert 20.

In an exemplary embodiment, the inventive insert may be made of polyethylene, for example, by blow molding, which is fast and economical to manufacture in high volume.

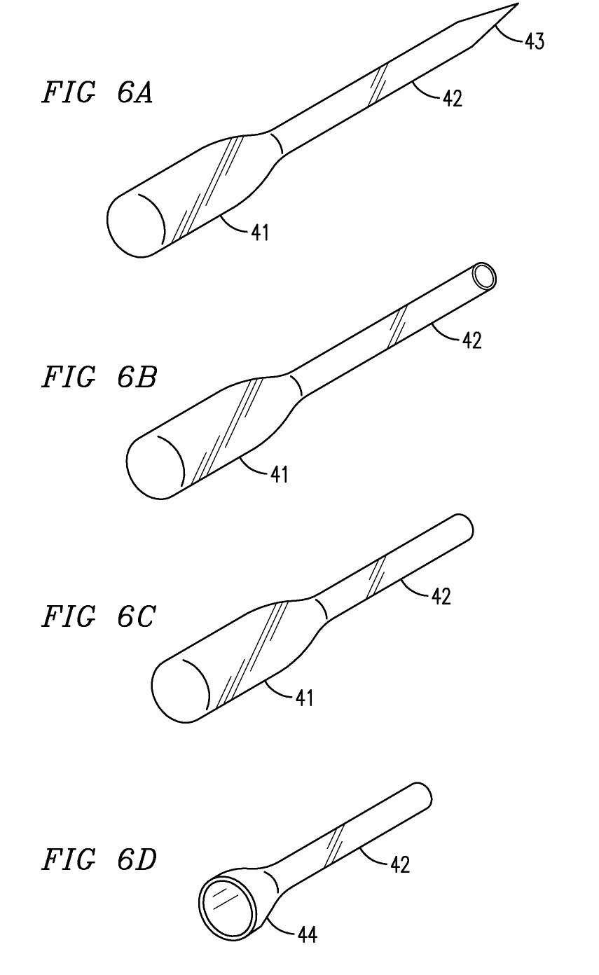

In a further example, as illustrated in FIG. 6A-6D, the inventive insert may be made from a disposable pipette. In this example, a pipette 40 with a bulb portion 41, a stem 42 and a tip portion 43 may be chosen as the starting point (FIG. 6A). The stem 42 may be, for example, 4.5 inch long stem with an internal diameter 0.25 inches. In a first step, illustrated in FIG. 6B, the tip 43 is cut off In a second step illustrated in FIG. 6C, the stem 42 may be sealed off, for example, by heat sealing, at a desired length from the bulb 41. For example, this length may be approximately 3.8 inches. In a third step illustrated in FIG. 6D, the bulb portion 41 is trimmed off to just retain a funnel-shaped portion 44. The steps illustrated above may be performed in any order and need not be performed in the order in which they are described in the present example. Alternate methods of manufacture may also be used.

The illustrated embodiments provide a salable consumable insert with practical utility for urinalysis and analysis of other biological and chemical samples. The embodiments provide a reliable solution for a common problem, namely repeated sample collection, which enables technicians to be more efficient with less interruption to workflow due to low volume samples.

While specific embodiments have been described in detail, those with ordinary skill in the art will appreciate that various modifications and alternative to those details could be developed in light of the overall teachings of the disclosure. For example, elements described in association with different embodiments may be combined. Accordingly, the particular arrangements disclosed are meant to be illustrative only and should not be construed as limiting the scope of the claims or disclosure, which are to be given the full breadth of the appended claims, and any and all equivalents thereof.

The following is a numbered list of non-limiting, illustrative embodiments of the inventive concepts disclosed herein:

1. An insert for a sample collection tube, comprising:

a generally hollow tubular body insertable into the sample collection tube, the tubular body being open at least at a first end,

wherein the tubular body of the insert has an internal cross-sectional area dimensioned to accommodate a width of test strip which comprises a plurality of test areas arranged along its length,

wherein the internal cross-sectional area of the tubular body is further dimensioned such that volume of 2.5 mL of a liquid sample inside the insert occupies a height which is at least sufficient to wet all of the test areas of the test strip when the strip is inserted lengthwise into insert containing the liquid sample.

2. The insert according to illustrative embodiments 1, wherein the tubular body has an internal diameter in the range of 0.2 to 0.3 inches.

3. The insert according to illustrative embodiments 1, wherein the height occupied by the volume of 2.5 mL of the liquid sample in the insert is in the range of 4.25 to 4.75 inches.

4. The insert according to illustrative embodiments 1, wherein the tubular body is sealed at a second end of the tubular body.

5. The insert according to illustrative embodiments 1, further comprising a funnel-shaped portion adjoining the first end of the tubular body.

6. The insert according to illustrative embodiments 1, wherein the insert is made of polyethylene.

7. The insert according to illustrative embodiments 1, further comprising a support structure for positioning the insert centrally within the sample collection tube.

8. The insert according to illustrative embodiments 1, wherein the liquid sample is a urine sample.

9. A collection kit for collecting a biological sample, comprising:

a sample collection tube, and

an insert according to claim 1, wherein the tubular body of the insert is narrower than the collection tube and is insertable into the collection tube for sample collection, whereby tubular body is positionable within the sample collection tube to receive the biological sample via its open end.

10. The collection kit according to illustrative embodiments 9, wherein the sample collection tube comprises a support structure for receiving the insert centrally within the sample collection tube.

11. A urine sample collection kit comprising:

a collection tube, and

an insert comprising a generally hollow tubular body with an open end, the tubular body being narrower than the collection tube and insertable into the collection tube,

wherein the tubular body of the insert has an internal cross-sectional area which is dimensioned with respect to the collection tube such that a volume of 2.5 mL of a urine sample inside the insert occupies a height which is at least equal to a height that would occupied by a 10 mL volume of the urine sample within the collection tube.

12. The urine sample collection kit according to illustrative embodiments 11, wherein the insert further comprises a funnel-shaped portion adjacent to the open end to receive the urine sample into the insert.

13. The urine sample collection kit according to illustrative embodiments 11, wherein the insert is sealed off at an end opposite to the open end.

14. The urine sample collection kit according to illustrative embodiments 11, wherein the insert comprises a support structure for positioning the insert centrally within the collection tube.

15. The urine sample collection kit according to illustrative embodiments 11, wherein the collection tube comprises a support structure for receiving the insert centrally within the collection tube.

16. A urinalysis kit comprising:

a collection tube,

an insert comprising a generally hollow tubular body narrower than the collection tube and insertable into the collection tube, and

a test strip comprising a plurality of analyte sensitive test areas, the test areas being arranged along a length of the test strip,

wherein the tubular body of the insert has an internal cross-sectional area dimensioned to accommodate the width of the test strip, such that the test strip is insertable lengthwise into the tubular body, and

wherein the internal cross-sectional area of the tubular body is further dimensioned such that volume of 2.5 mL of a urine sample inside the insert occupies at least a height which is sufficient to wet all of the test areas of the test strip, when the strip is inserted lengthwise into the insert containing the urine sample.

17. The urinalysis kit according to illustrative embodiments 16, wherein the tubular body of the insert has an internal diameter in the range of 0.2 to 0.3 inches.

18. The urinalysis kit according to illustrative embodiments 16, wherein each of the test areas of the test trip comprises a reagent pad which is capable of undergoing a color change in response to contact with the urine sample.

19. The urinalysis kit according to illustrative embodiments 16, wherein one end of the insert is funnel-shaped and configured for receiving the urine sample in the insert.

20. The urinalysis kit according to illustrative embodiments 16, wherein the height occupied by the volume of 2.5 mL of the urine sample in the insert is in the range of 4.25 to 4.75 inches.

21. A urine sample collection container comprising:

a unitary elongated body comprising an open first end configured to receive a urine sample and a closed second end,

the unitary elongated body having an outer surface defining an outer cross-sectional area and an internal cavity defining an internal cross-sectional area narrower than the outer cross-sectional area,

wherein the internal cross-sectional area of the cavity is dimensioned to accommodate a width of test strip which comprises a plurality of test areas arranged along its length,

wherein the internal cross-sectional area of the cavity is further dimensioned such that volume of 2.5 mL of the urine sample in the cavity of the container occupies a height which is at least sufficient to wet all of the test areas of the test strip when the strip is inserted lengthwise into the cavity of the container containing the urine sample.

What is claimed is:

1. An insert for a sample collection tube, comprising:
a generally hollow tubular body insertable into the sample collection tube, the tubular body being open at least at a first end,
wherein the tubular body of the insert has an internal cross-sectional area dimensioned to accommodate a width of test strip which comprises a plurality of test areas arranged along its length,
wherein the internal cross-sectional area of the tubular body is further dimensioned such that volume of 2.5 mL of a liquid sample inside the insert occupies a height which is at least sufficient to wet all of the test areas of the test strip when the strip is inserted lengthwise into insert containing the liquid sample.

2. The insert according to claim 1, wherein the height occupied by the volume of 2.5 mL of the liquid sample in the insert is in the range of 4.25 to 4.75 inches.

3. The insert according to claim 1, further comprising a funnel-shaped portion adjoining the first end of the tubular body.

4. The insert according to claim 1, further comprising a support structure for positioning the insert centrally within the sample collection tube.

5. The insert according to claim 1, wherein the liquid sample is a urine sample.

6. A collection kit for collecting a biological sample, comprising:
a sample collection tube, and
an insert according to claim 1, wherein the tubular body of the insert is narrower than the collection tube and is insertable into the collection tube for sample collection, whereby tubular body is positionable within the sample collection tube to receive the biological sample via its open end.

7. The collection kit according to claim 6, wherein the sample collection tube comprises a support structure for receiving the insert centrally within the sample collection tube.

8. A urine sample collection kit comprising:
a collection tube, and
an insert comprising a generally hollow tubular body with an open end, the tubular body being narrower than the collection tube and insertable into the collection tube,
wherein the tubular body of the insert has an internal cross-sectional area which is dimensioned with respect to the collection tube such that a volume of 2.5 mL of a urine sample inside the insert occupies a height which is at least equal to a height that would occupied by a 10 mL volume of the urine sample within the collection tube.

9. The urine sample collection kit according to claim 8, wherein the insert is sealed off at an end opposite to the open end.

10. The urine sample collection kit according to claim 8, wherein the insert comprises a support structure for positioning the insert centrally within the collection tube.

11. The urine sample collection kit according to claim 8, wherein the collection tube comprises a support structure for receiving the insert centrally within the collection tube.

12. A urinalysis kit comprising:
a collection tube,
an insert comprising a generally hollow tubular body narrower than the collection tube and insertable into the collection tube, and
a test strip comprising a plurality of analyte sensitive test areas, the test areas being arranged along a length of the test strip,
wherein the tubular body of the insert has an internal cross-sectional area dimensioned to accommodate the width of the test strip, such that the test strip is insertable lengthwise into the tubular body, and
wherein the internal cross-sectional area of the tubular body is further dimensioned such that volume of 2.5 mL of a urine sample inside the insert occupies at least a height which is sufficient to wet all of the test areas of the test strip, when the strip is inserted lengthwise into the insert containing the urine sample.

13. The urinalysis kit according to claim 12, wherein the tubular body of the insert has an internal diameter in the range of 0.2 to 0.3 inches.

14. The urinalysis kit according to claim 12, wherein each of the test areas of the test trip comprises a reagent pad which is capable of undergoing a color change in response to contact with the urine sample.

15. A urine sample collection container comprising:
a unitary elongated body comprising an open first end configured to receive a urine sample and a closed second end,
the unitary elongated body having an outer surface defining an outer cross-sectional area and an internal cavity defining an internal cross-sectional area narrower than the outer cross-sectional area,
wherein the internal cross-sectional area of the cavity is dimensioned to accommodate a width of test strip which comprises a plurality of test areas arranged along its length,
wherein the internal cross-sectional area of the cavity is further dimensioned such that volume of 2.5 mL of the urine sample in the cavity of the container occupies a height which is at least sufficient to wet all of the test areas of the test strip when the strip is inserted lengthwise into the cavity of the container containing the urine sample.
